(19) United States
(12) Patent Application Publication
(10) Pub. No.: US 2015/0328356 A1
(43) Pub. Date: Nov. 19, 2015

(54) SPOUTING OR SPITTING CETACEAN AUTOMATIC AIR FRESHENER

(71) Applicant: Kevin Laron Howell, Deltona, FL (US)

(72) Inventor: Kevin Laron Howell, Deltona, FL (US)

(21) Appl. No.: 14/280,597

(22) Filed: May 17, 2014

(51) Int. Cl. A61L 9/14; B65D 83/26

(52) U.S. Cl. CPC A61L 9/14; B65D 83/262

(57) ABSTRACT

The purpose or the art is to freshen the air in living spaces via an automatic air freshener designed to appear like a spouting or spitting cetacean. The invention consists of a manufactured cetacean which may or may not house the inner working parts; an optional spherical mid-section which houses the working parts consisting of a bracket that holds an aerosol spray can, a motor, gears, a motor controller, batteries, and a tube. The motor drives a set of gears which engage a fan shaped lever type gear connected to an actuating bolt that depresses the nozzle of the aerosol spray can. These parts are housed in the manufactured cetacean's body or the art's mid-section. The invention will be supported by a base. The working parts provide the mechanisms by which the spouting a spitting cetacean expels air freshener through its blow hole or mouth via a tube connected to either orifice.

### BACKGROUND OF THE INVENTION

1. Field of the Invention

The invented art is a remote controlled automatic air freshener.

2. Description of the Invention

The Spouting or Spitting Cetacean Automatic Air Freshener is an artful center piece for interior or exterior living spaces. The manufactured art imitates life in the aspect of producing a directed spray stream of air freshener on timed intervals as spouting cetaceans blow water and mucus from their blow hole or mouth. The art consists of three main parts: 1.) a hollow manufactured cetacean with an internal tube connected to its blow hole or mouth to allow air freshener fluid to be carried to and expelled from one of the two orifices; 2.) an optional hollow spherical mid-section which houses the art's internal mechanical elements; and 3.) a base to support the invention. Production of the art will be sourced from artisans skilled in working with glass, ceramic, metal, plastic, marble, and wood.

### SUMMARY OF THE INVENTION

The current invention is to provide an aromatic spray mist which freshens the air in living spaces. The invention's novelty in its representation of cetacean activity would appeal to those passionate about cetaceans and increasing public awareness of the continued need to insure that these mammals have a safe environment in which they will flourish. The cetacean will be crafted from ceramic, porcelain, terracotta, plastic, brass, glass, metal, stone, or wood. Central to the art's design is a tube connected to the art's blow hole or mouth allowing for a pressurized air freshener liquid to be expelled from either of the orifices depending on the design of the particular piece of art (i.e., type of cetacean).

The working parts of the air freshener which enable the invention to spout or spit air freshener consists of a rotary motor; a gear assembly; and a rocker arm type lever gear that transfers rotary torque into linear force applying downward pressure on the nozzle of an aerosol spray can. All of which are secured to a bracket. The air freshener liquid is released into the internal tube attached to a hollow nozzle actuating bolt that passes through the rocker arm that delivers the force to depress the aerosol can spray nozzle. The tube then carries the liquid to the manufactured cetacean's blow hole or mouth to be expelled into the surrounding environment. The internal parts may be housed in the cetacean portion of the art, if the design does not consist of a mid-section.

### BRIEF DESCRIPTION OF THE DRAWINGS

FIG. 1. Is the cover page which depicts a set of manufactured dolphins 1 that serve as an example of the spouting version of the automatic air freshener; detail 2 is the optional mid-section which houses the internal working parts of the automatic air freshener; 3 is the base of the invention. The trim 4 around the spherical mid-section houses the on/off button 5 and the motion and remote sensors 5.

FIG. 2 depicts an orca 6 which is representative of one of the various types of cetaceans that the art entails.

FIG. 3 is across-sectional view of the orca 7; the internal spray tube 8 which carries air freshener to the cetacean's blow hole; and the optional mid-section 9 with an opening on the bottom which allows for the removal and replacement of the aerosol can via the internal bracket 13.

FIG. 4 is the sectional view of the spitting dolphin 10 version of the invention; the internal tube 11; and the spray nozzle tip 12 located in the dolphin's open mouth.

FIG. 5 is the rear view of the internal bracket 13; the telescopic channel slide 14; the rotary motor 15; the housing for the motor controller and batteries 16; and the spring loaded latch 17.

FIG. 6 shows the bottom view of the internal bracket with telescoping channel slide 18 and the arms which secure the bracket to the mid-section and base 18; detail 19 depicts the air freshener's manual controls and battery compartment; 20 is the bottom view of the spring loaded latch.

FIG. 7 is a detailed view of the spring loaded latch 21.

FIG. 8 is a view of the detached rocker arm type lever gear 22; the internal tube 24 attached to the hollow nozzle actuator bolt 23 and threaded through the lever gear 22, a washer 26, and a nut 25.

FIG. 9 is a cross-sectional view of the spherical mid-section which houses the internal bracket 27 and a latch 28 which secures the aerosol spray can to the carrier attached to the telescopic channel slide 14. FIG. 9 also shows the location of the rocker arm type lever gear 29.

FIG. 10 is an example of the orca 30 embodiment of the art which houses the internal working parts in the manufactured cetacean's body.

FIG. 11 is a much smaller internal bracket relative to that shown in FIG. 5. This smaller bracket 31 does not have a telescoping channel. Instead the bracket has interlocking u-shaped grooves that allow the front portion of the bracket 32 which carries the aerosol can to slide into position and engage the hollow actuating bolt 34 shown in FIG. 13. Detail 33 shows the rotary motor and rocker arm gear assembly.

FIG. 12 is a cross-sectional view of the baseless orca version of the art 34.

FIG. 13 is a detailed view of the rocker arm type lever 35 for the small bracket gear assembly. 36 and 37 are the smaller nut and washer respectively.

### DETAILED DESCRIPTION OF THE INVENTION

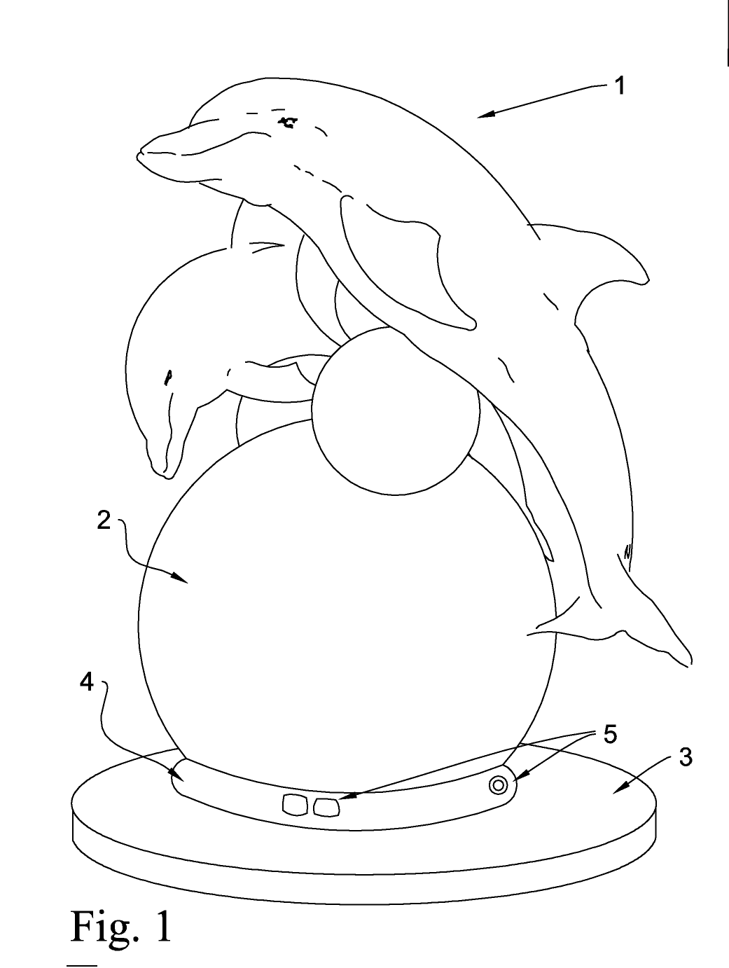

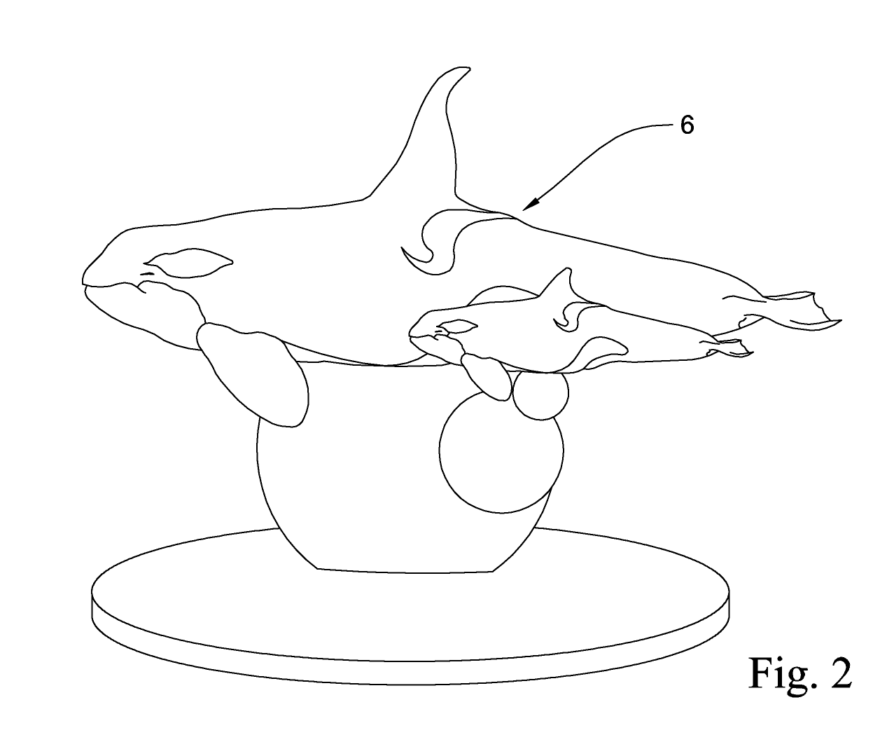

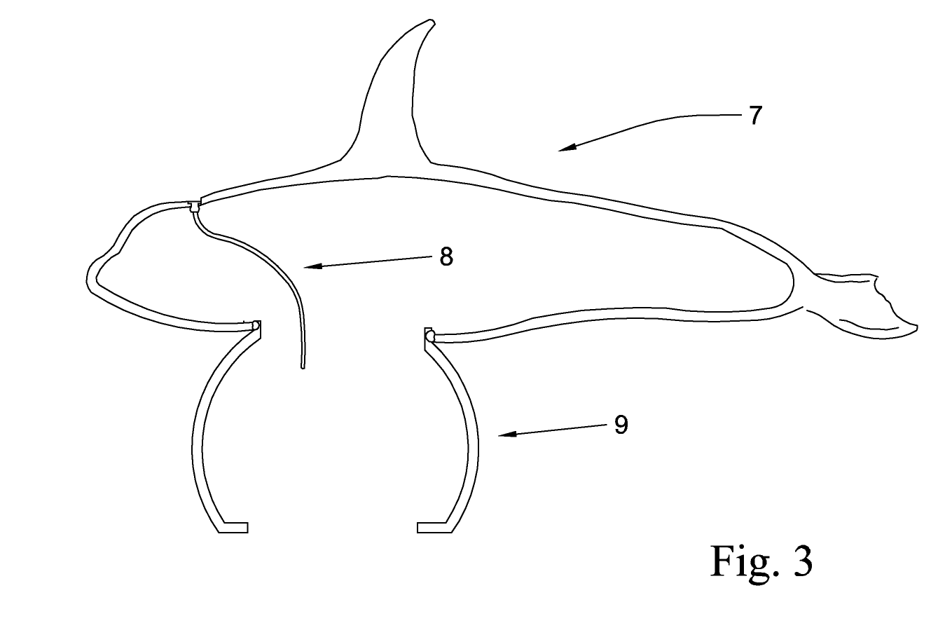

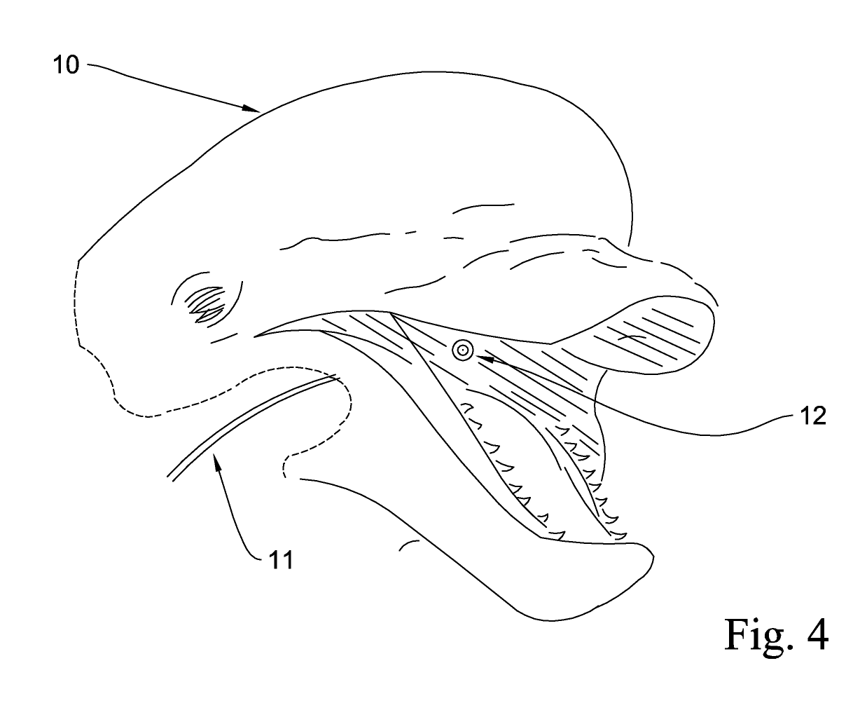

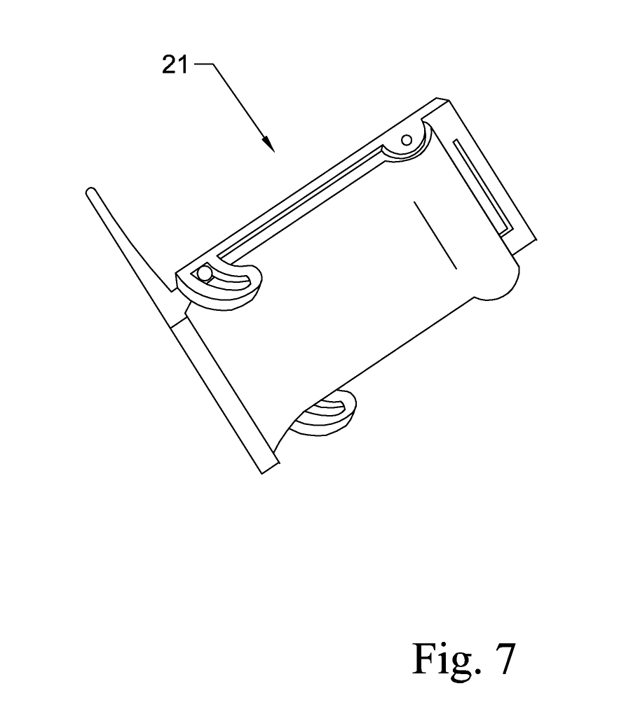

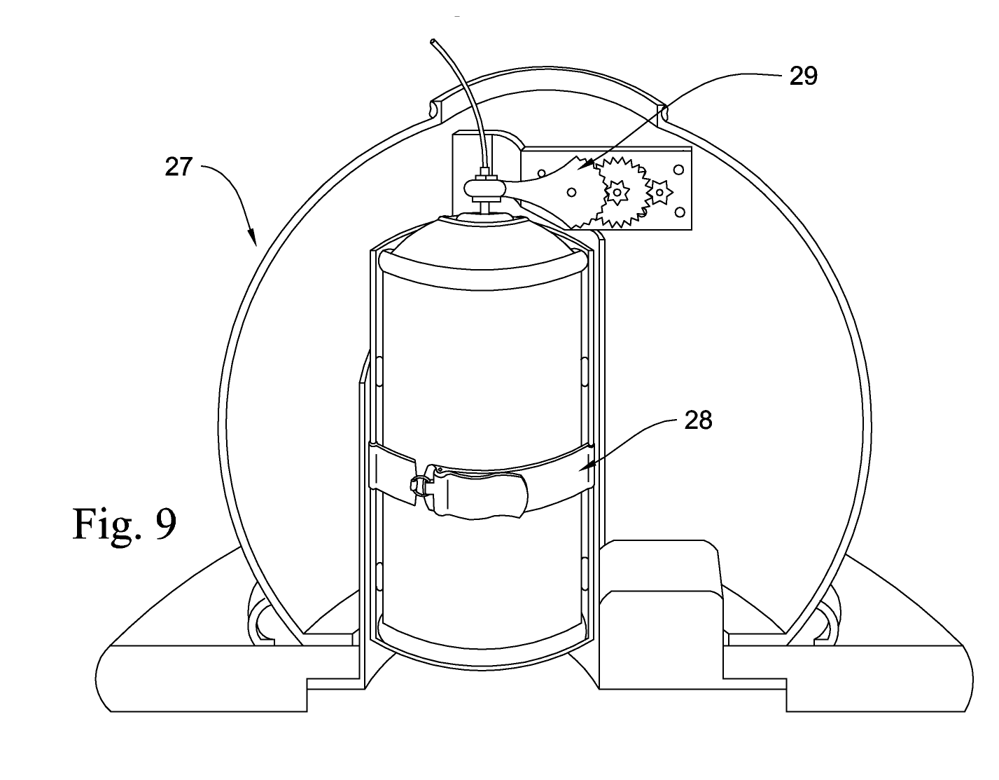

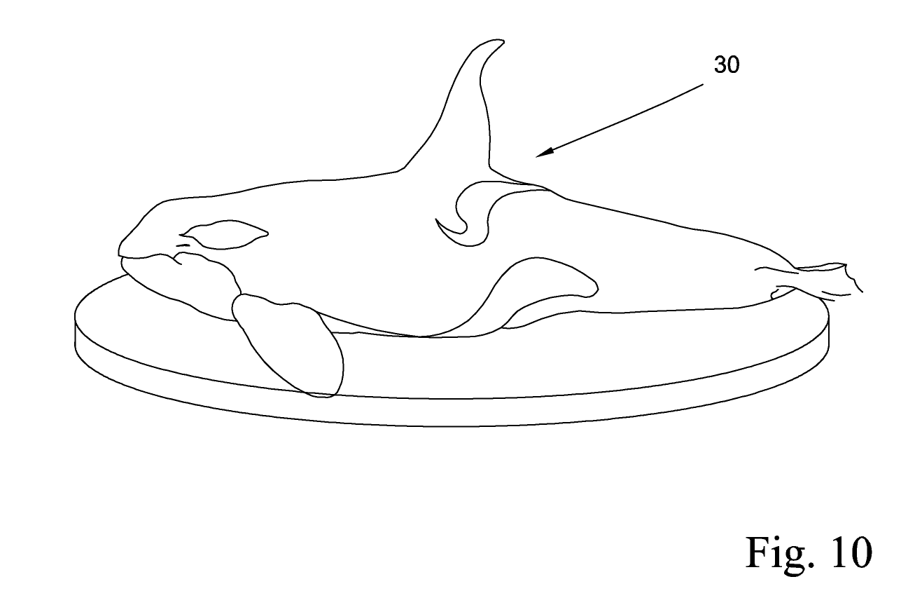

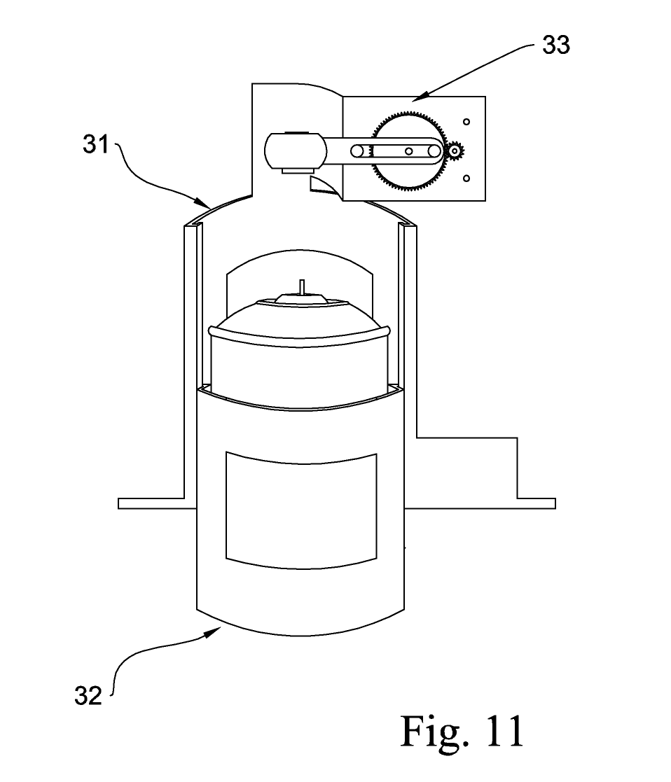

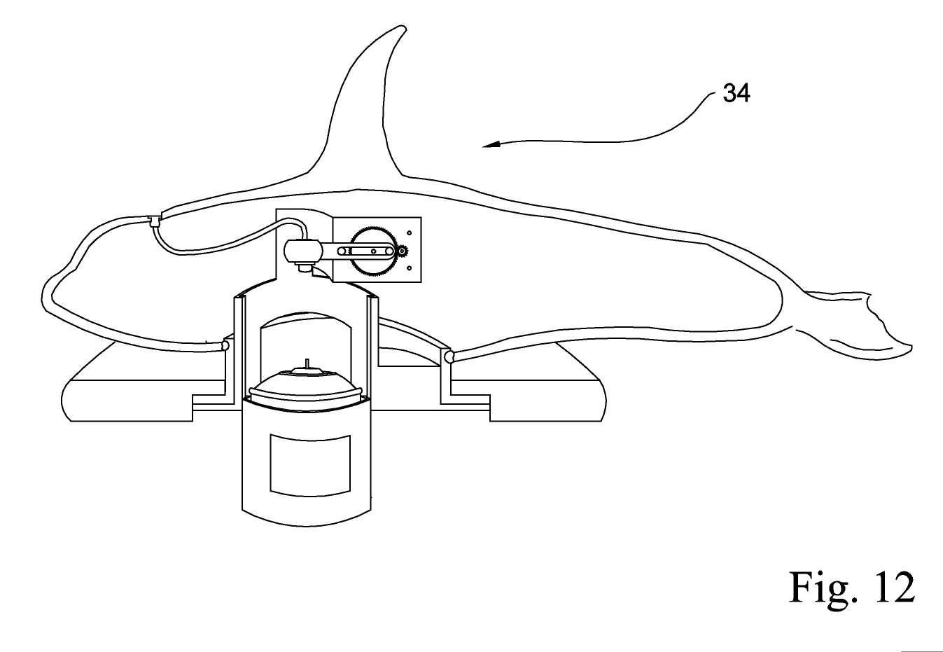

The invention is an automatic air freshener in the form of a spouting or spitting cetacean. The invention consists of the following embodiment with reference to FIGS. 1 to 13: FIGS. 1, 2, 4, and 10 are representatives of the desired embodiments of the art expressed as a set of dolphins, a killer whale, and a spitting dolphin. The renderings of the near dolphin 1 and the killer whale 6 and 30 expressions of the art serve the function of releasing air freshener into the surrounding area via their blow-holes. The sectional view rendering of a spitting dolphin detail 10 provides an example of how the invention sprays air freshener through the dolphin's mouth. The working parts of the invention are housed in an optional spherical mid-section 2 representing water or in the body of the manufactured cetacean FIG. 12.

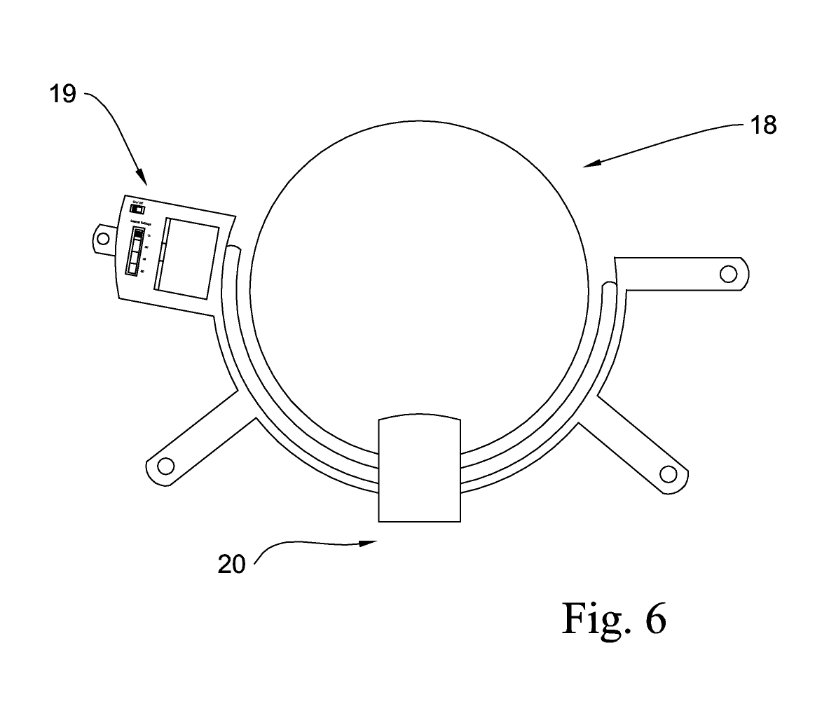

The invention's internal working parts are configured as follows: an internal custom bracket 13 is secured to the optional mid-section 27 and base 3. This allows for the replacement of an aerosol spray can. Attached to the top of the bracket is a rotary motor 15 whose transmission turns a drive gear which engages an idle gear which in turn engages the lever gear 22 or rocker arm type gear 35 depending on the model of the art. The lever gear 22 or rocker arm type gear 35 both have the internal spray tube 24 threaded through and attached to the actuating bolt 34 that provides downward force which releases air freshener liquid into the internal tube and out of the cetacean's blow hole or mouth. A motor controller located in its housing 16 controls the intervals in the rotation of the motor. The air freshener is battery operated with manual and remote controls FIG. 1 and detail 19 of FIG. 6. The invention is designed to have the flexibility to be personalized by the end user. The design allows for the end user to choose between types of materials for representations of cetaceans, bases, trim, and an option to omit the mid-section.

The embodiments of the invention in which an exclusive privilege is claimed are defined as follows:

1-3. (canceled)

4. a manufactured automatic air freshener which comes in the form of all types of cetaceans; the spouting or spitting action of the cetacean air freshener in the form of releasing a spray mist of air freshener from the mouth or blow hole.

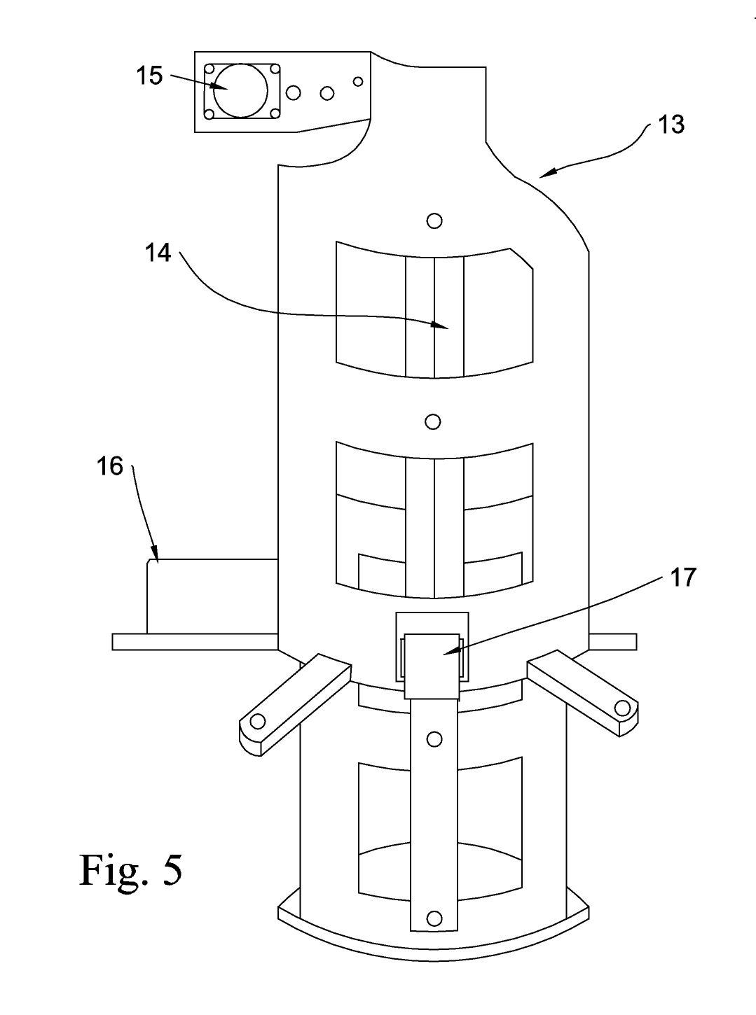

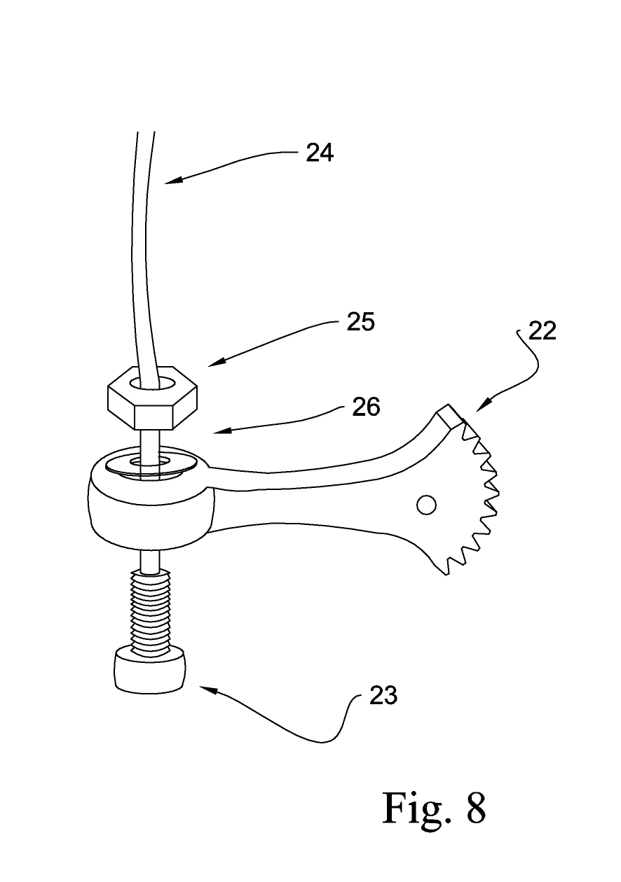

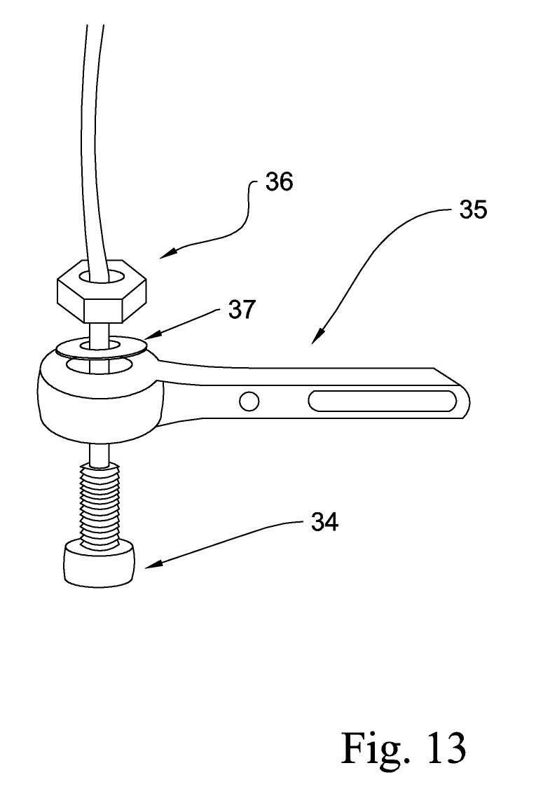

5. the composition of the internal working parts for the large and small brackets FIGS. 5, 6, and 11; the design of the lever gear and actuating bolt FIGS. 8 and 13
